(19) United States
(12) Patent Application Publication
(10) Pub. No.: US 2025/0204552 A1
(43) Pub. Date: Jun. 26, 2025

(54) A MICROBIAL CELL EXTRACT, METHOD FOR OBTAINING SAID MICROBIAL CELL EXTRACT AND USE OF SAID MICROBIAL CELL EXTRACT

(71) Applicant: FUMI Holding B.V., Wageningen (NL)

(72) Inventors: Edgar Suarez GARCIA, Wageningen (NL); Corjan VANDENBERG, Wageningen (NL)

(21) Appl. No.: 18/848,360

(22) PCT Filed: Mar. 17, 2023
(86) PCT No.: PCT/EP2023/056907

§ 371 Date: Sep. 18, 2024

(30) Foreign Application Priority Data
Mar. 18, 2022 (NL) 2031322

(51) Int. Cl. A23K 10/16; A23K 40/10; A23K 50/75; C12N 1/18; C12N 1/20; C12R 1/01; C12R 1/85

(52) U.S. Cl. CPC A23K 10/16; A23K 40/10; A23K 50/75; C12N 1/185; C12N 1/205; C12R 2001/01; C12R 2001/85

(57) ABSTRACT

The invention relates to a method for producing a microbial cell extract. The invention further relates to a microbial cell extract obtained by or obtainable by said method. The invention further relates to the use of said microbial cell extract, with applications in diet formulations for animals.

### TECHNICAL FIELD

The invention relates to a method for producing a microbial cell extract. The invention further relates to a microbial cell extract obtained by or obtainable by said method. The invention further relates to the use of said microbial cell extract, with applications in diet formulations for animals.

### BACKGROUND OF THE INVENTION

The world population is projected to further increase, consequently driving an increase in the demand for plant based and animal-based products, therefore there is a need to develop sustainable farming practices to minimise the environmental impact of generating these products. For the case of animal-based products, current farming practices are under considerable scrutiny due to the use of refined plant proteins, antibiotics, synthetic feed supplements and growth promoting additives. There are also problems with the poor hygienic conditions and overcrowded spaces in which animals are grown in traditional practices.

Novel sustainable practices must include the implementation of diets based on ingredients that can contribute to the health and well-being of animals, while simultaneously improving feed conversion rates (FCR). It is also desirable that the feed formulations contain ingredients sourced with minimal carbon footprint to mitigate the effects of global climate change.

In this regard, microbial biomass offers several advantages. Firstly, it is produced in substantial quantities in the biotechnology industry, either as a main product or as a by-product from fermentation processes. When generated as a by-product, it is then usually either disposed of, used as an energy source, or used as a feed supplement or raw material for other processes. It is therefore important to develop new valorisation strategies to make use of such abundant biomass. Secondly, when said biomass is produced in bioreactors under controlled conditions, the environmental footprint, including land and water usage, is significantly advantageous. Thirdly, due to its structural diversity and composition, microbial biomass can provide a wide spectrum of functional ingredients including proteins, peptides, (poly)saccharides, lipids, fatty acids, vitamins amongst other others. Of particular importance are the cell walls and membranes of microbial cell biomass, which are rich in non-digestible polysaccharides (NDP), which have been extensively shown in the scientific literature to bring important health and nutritional benefits to animals. This is because NPD selectively stimulates health promoting microbial communities in the gut, which then manifests as superior growth rates, better assimilation of nutrients, and stronger immune responses, amongst other advantageous effects.

Examples of NDP include insulin and galacto-oligosaccharides, but perhaps the most studied case is the group of polysaccharides present in the cell wall of yeast and fungi: glucans, mano-oligosaccharides and chitin. The β-1,3 and β-1,6 glucanic structure of yeast have shown to have remarkable immunomodulating properties in humans and animals (Avramia and Amariei, 2021). Algae biomass have received less attention and most applications involve the use of whole or weakened biomass to enhance nutrient accessibility, protein and fat content, resulting in superior weight gain and feed conversion in feed trials (Agboola et al., 2019). As such, novel methods, which can also be applied to a broader range of microbes, to prepare functional NDP are needed.

β-glucans from yeast have been traditionally produced by chemical and energy intensive processes involving cell disintegration followed by a series of separation and purification steps. Cell disintegration is conducted by biological, chemical and physical processes. Biological processes include autolysis, autophagy and enzymatic hydrolysis (proteases and glucanases). Chemical processes include chemical hydrolysis, treatment with solvents and plasmolysis. Among the physical methods, high pressure homogenization, bead milling, osmotic shocks, ultrasounds, electric fields and freeze-thaw cycles, or combinations, are the most common (Avramia and Amariei, 2021).

Upon cell disintegration, the soluble compounds are often removed using methods such as centrifugation and filtration, leaving an insoluble fraction from which additional purification steps are needed. The most common purification method is a sequence of aqueous, strong alkaline-high temperature and acid extraction steps. Alkaline conditions at high temperature are needed to solubilize mannoproteins and other impurities embedded in the polymeric structure, while (acetic) acid is used to solubilize glycogen. Other chemicals used for the extraction of β-glucans are NaOH/HCl, NaOH/CH3COOH, NaOH/NaClO and NaOH+NaClO/DMSO. Organic acids such as ethanol, and supercritical solvents such as CO₂, may also be used to reduce the content of lipids and therefore to increase the purity of the extracted β-glucan fraction. Enzyme hydrolysis, in particular with nucleases, proteases, lipases, and glucanases, is also commonly used to selectively hydrolyse the polymer chains in the cell walls, and other unwanted cell structures. Other innovative processes have been reported, for example using ionic liquids, resulting in excellent yields but affecting the structure of the glucans. A complete overview of processes and extraction methods developed for β-glucan extraction from yeast is presented by Avramia and Amariei (2021).

Animal feed products have been produced from microbial biomass that have desirable properties such as a prebiotic effect or an improvement in FCRs, however, such products require complex methods. For example, EP3685681A1 describes a method for preparing a yeast product and its use as a prebiotic in feed and food preparations. The method includes the steps of cell disintegration by thermal plasmolysis, separation and treatment with enzymes to yield a protein rich insoluble fraction from a soluble fraction and where the yield product has a β-glucan content >15% and low RNA content. EP2170359 presents a method that involves enzyme hydrolysis, alkaline-acid extraction and high temperature incubation to yield a yeast fraction with at least 37% β-glucan and mannan content. Similarly, EP3266863A1 claims a method where an enzyme proteolytic reaction, under alkaline conditions is used to produce a yeast product containing 20-24% glucans, which can be used as a feed and food supplement. EP3485743A1 describes a food product containing glucans from yeast (produced from a glucan rich starting material, subjected to enzymatic hydrolysis with endoglucanases); said product can be used to enhance feed conversion ratios, body weight and immune status of animals. PT1965809E claims a method for thermally processing seaweed (75° C.) at low pH followed by clarification and filtration to obtain a soluble fraction and its use in pharmaceutical and feed compositions to improve the gut health of humans and animals. US2018168190A1/WO2019160599A1 describes a feed composition which includes soy and corn in combination with added whole Euglena cells or lysed Euglena cells, with at least 30% β-1,3 glucan. Such compositions further include immune response enhancer compounds added to the composition, such as vitamins and polyunsaturated fatty acids. US2006286205A1 presents a method for hydrolysing and pre-treating oleaginous microbial biomass of the groups algae, protists, bacteria and fungi, and for producing an emulsified nutritional product. Although this prior art describes the production of desirable products, a drawback is the intense and complex methods required for production. Furthermore, in many cases the specific microbial biomass that can be utilised is often limited.

There is therefore a need to develop novel methods that can be applied to a broader range of microbial biomass than covered by previous methods. There is also a need to develop methods that are simpler than those that have been established previously, in particular without requiring intensive chemical, thermal or enzymatic treatments, and that produce functional ingredients with prebiotic activity, even when at low purities. The present invention addresses this need.

### SUMMARY OF THE INVENTION

In a first aspect of the invention, there is provided a method of preparing a microbial cell extract, the method comprising:
a) providing a microbial biomass in an aqueous alkaline suspension;
b) mechanically disintegrating the microbial biomass at a temperature below 35° C. to produce a disintegrated biomass, wherein the disintegrated biomass comprises or consists of a population of soluble compounds and suspended fragments characterized by a bimodal distribution and a particle size distribution (psd) at an average of around D50<4.5 μm;
c) separating the disintegrated biomass into a light fraction and a heavy fraction comprising fragments of different sizes suspended in an aqueous mixture, wherein the light fraction comprises fragments in the range 0.1-3 μm and the heavy fraction comprises fragments above 1 μm, wherein the volumetric ratio of the heavy fraction to the starting disintegrated biomass is 0.65 or more; and
d) selecting only said heavy fraction.

In one embodiment, the microbial biomass is prepared in an aqueous alkaline suspension at a pH of around 9.

In another embodiment, the mechanical disintegration step is carried out by bead milling or high-pressure homogenization. Preferably, the disintegration step is carried out at a pH in the range of 6 to 9, and at a temperature in the range of 10-30° C.

In another embodiment, the disintegrated biomass is separated using centrifugation, decantation or filtration. Preferably, centrifugation is carried out at around 4000×g for around 10 minutes. In one embodiment, the disintegrated biomass is separated into a light fraction having a multimodal psd in the range of 0.1 to 3 μm and the disintegrated biomass is separated into a heavy fraction having a multimodal psd in the range of 1 to 15 μm. In one embodiment, the heavy fraction comprises fragments above 1 μm, wherein preferably the volumetric ratio of the heavy fraction to the starting disintegrated biomass is >0.9.

In one embodiment, the microbial cell extract has a water holding capacity of at least twice its own dry weight. In another embodiment, the microbial cell extract has an oil holding capacity of at least 1.5 times its own dry weight. In another embodiment, the microbial cell extract has a dry weight β-glucan content of around 20% and above. In another embodiment, the microbial cell extract has a dry weight RNA content of around 5% or less. In another embodiment, the microbial cell extract has a dry weight protein content between 30-60%, preferably around 36% and/or a dry weight carbohydrate content of about 30 to 60%, preferably around 43% and/or a dry weight dietary fiber concentration between 30 and 40%, preferably around 34%.

In another embodiment, the method further comprises
a) mechanically disintegrating the heavy fraction, wherein disintegration is carried out at a pH in the range of 9 to 11, preferably a pH of around 11;
b) diluting the disintegrated heavy fraction to obtain a dry weight (DW) content of 2.5% or more;
c) subjecting the disintegrated and diluted heavy fraction to a solid-liquid separation process to separate the disintegrated heavy fraction into a first fraction with a particle size distribution (psd) at around D90<0.27 μm and a second fraction with a particle size distribution (psd) at an average of around D50>18 μm; and
d) selecting said second fraction.

In one embodiment, this microbial cell extract (second fraction described above) has a water holding capacity of at least 10 times its own dry weight. In another embodiment, this microbial cell extract has a dry weight β-glucan content of 40% and above. In another embodiment, this microbial cell extract has a dry weight RNA content of 2% or less. In another embodiment, this microbial cell extract has a gel hardness of at least 2N.

In another embodiment of the aspect of the invention, the method further comprises subjecting the microbial cell extract to one or more additional processes, selected from filtration, adsorption, isoelectric precipitation, coagulation and solvent extraction. In another embodiment, the method further comprises concentrating and/or drying the microbial cell extract to reduce the water content to around 5%. Preferably, the concentrating and/or drying the microbial cell extract is carried out by filtration, evaporation, freeze concentration, pervaporation, sublimation, drying by spray drying or drum drying.

The microbial biomass used in the present invention can be obtained from several microbial types, including microalgae, yeast, bacteria and fungi. Examples of genus from which the microbial biomass may be derived for the microbial extract may be produced are Saccharomyces and Pichia (yeast), Tetraselmis, Chlorella, Arthrospira (algae), Fusarium (fungi), Methylobacterium (bacteria) and Lactobacillus (bacteria). Preferably the microbial biomass is derived from yeast, more preferably from the genus Saccharomyces and/or Pichia. Yeasts which may be used in the present invention include Saccharomyces, such as S. cerevisiae, S. chevalieri, S. boulardii, S. bayanus, S. italicus, S. delbrueckii, S. rosei, S. micro-ellipsodes, S. carlsbergensis, S. bisporus, S. fermentati, S. pastorianis, S. rouxii, or S. uvarum; a yeast belonging to the genus Schizo-saccharomyces, such as S. japonicus, S. kambucha, S. octo-sporus, or S. pombe; a yeast belonging to the genus Hansenula, such as H. wingei, H. arni, H. henricii, H. americana, H. canadiensis, H. capsulata, or H. polymorpha; a yeast belonging to the genus Candida, such as C. albicans, C. utilis, C. boidinii, C. stellatoidea, C. famata, C. tropicalis, C. glabrata, or C. parapsilosis; a yeast belonging to the genus Pichia, such as P. pastoris, P. kluyveri, P. polymorpha, P. barkeri, P. cactophila, P. rhodanensis, P. cecembensis, P. cephalocereana, P. eremophilia, P. fermentans, or P. kudriavzevii; a yeast belonging to the genus Kluyveromyces, such as K. marxianus; and a yeast belonging to the genus Torulopsis, such as T. bovina, or T. glabrata.

In another aspect of the invention, there is provided a microbial cell extract obtained or obtainable by the method of the invention.

In another aspect of the invention, there is provided a method for preparing an animal diet formulation, the method comprising combining the microbial cell extract of the invention in a diet formulation using extrusion, palletization, blending or coating.

In one embodiment, the animal diet formulation comprises 0.01% by weight or more of the microbial cell extract.

In another aspect of the invention, there is provided an animal diet formulation obtained or obtainable by the method of the invention.

In another aspect of the invention, there is provided a microbial cell extract, the extract comprising microbial cell fragments between 1 and 15 μm suspended in an aqueous mixture.

In one embodiment, the extract is characterized by a dry weight β-glucan content of 20% or more and a dry weight RNA content of 5% or less. In another embodiment, the extract is characterized by a dry weight β-glucan content of 40% or more and a dry weight RNA content of 2% or less. In another embodiment, the extract has a water holding capacity of at least twice its own dry weight. In another embodiment, the extract has a water holding capacity of at least ten times its own dry weight. In another embodiment, the extract has an oil holding capacity of at least 1.5 times its own dry weight.

In another aspect of the invention there is provided an animal diet formulation comprising 0.01% by weight or more of the microbial cell extract of any of the invention.

In another aspect of the invention, there is provided the use of the microbial cell extract of the invention or the animal feed formulation of the invention for use as a pre-biotic.

In another aspect of the invention there is provided a method of decreasing feed conversion rates and/or increasing the body weight of an animal, the method comprising feeding the microbial cell extract of the invention or the animal feed formulation of the invention to the animal.

In another aspect of the invention there is provided, a method of increasing the litter quality of an animal, the method comprising feeding the microbial cell extract of the invention or the animal feed formulation of the invention to the animal. Preferably, the animal is livestock. Preferably, the animal is not a human. In one embodiment, body weight of the animal is increased by 20% or more compared to an animal that is not fed the microbial cell extract of the invention or the animal feed formulation of the invention.

### DESCRIPTION OF THE FIGURES

The invention is further described in the following non-limiting figures:

FIG. 1 shows a bimodal distribution of disrupted yeast cells and corresponding psd.

FIG. 2 shows the psd of the light and heavy phase.

FIG. 3 shows the cumulative feed conversion ratios for several treatments and growth phases

FIG. 4 shows the litter scores for several diets at day 21 and at the end of the trial (day 43). Sections with dots and diagonal lines with a positive gradient represent higher scores (worse quality), while sections with negative gradient diagonal lines, and block colour sections represent better quality (lower score).

FIG. 5 shows the gelation hardness, water and oil holding capacities of extracts produced after extended disintegration at different pH values.

FIG. 6 shows the D[3,2] and D50 of the population of fragments during mechanical disintegration at different pH values.

FIG. 7 shows the β-glucan content of microbial extracts of the invention compared to a reference process. MlcExt1+W is the microbial extract plus an aqueous washing step.

FIG. 8 shows the psd of heavy and light phase after long-term/extended bead milling at pH 11, dilution and centrifugation.

FIG. 9 shows the D10, D50, D90 for a bimodal distribution based on volume.

FIG. 10 shows enrichment of particles from a disrupted suspension with a population of fragments (FIG. 10a) and separation into light and heavy phase under high centrifugal force (FIG. 10b), medium centrifugal force (FIG. 10c) and low centrifugal force (FIG. 10d). Phase split is indicated with a horizontal line.

FIG. 11 shows psd of yeast biomass (circles), disrupted biomass (squares), the fraction enriched in small fragments (triangles) and the fraction enriched in large fragments (inverted triangles) according to the invention.

FIG. 12 shows the psd of the light phase after cell disintegration and centrifugal separation at several intensities (g forces): high intensity of 20000×g for 15 minutes (triangles), medium intensity of 4000×g for 15 minutes (squares) and low intensity of 1000×g for 5 minutes (circles).

FIG. 13 shows psd of the disintegrated biomass, light fraction (EESF) and heavy fraction (EELF)) derived from Methylobacterium spp. according to the present invention.

### DETAILED DESCRIPTION OF THE INVENTION

The following embodiments apply to all aspects of the present invention.

The present invention will now be further described. In the following passages, different aspects of the invention are defined in more detail. Each aspect so defined may be combined with any other aspect or aspects or embodiment or embodiments unless clearly indicated to the contrary. In particular, any feature indicated as being preferred or advantageous may be combined with any other feature or features indicated as being preferred or advantageous.

The following definitions are used in the present description and claims to define the stated subject matter. Other terms not cited below are meant to have the generally accepted meaning in the field.

“Drying” as used in the present description means reducing the moisture content. The term drying includes partial drying wherein moisture may remain after drying in a reduced amount, which can also be seen as concentrating.

“Dry weight (DW)” and “dry cell weight” as used in the present description mean weight determined in the relative absence of water. For example, reference to microbial biomass as comprising a specified percentage of a particular component by dry weight means that the percentage is calculated based on the weight of the biomass after substantially all water has been removed.

“Disruption” as used in the present description in the context of microbial cells is also referred to as “lysing” and means opening the cells to release cytoplasmic compounds (also referred to as the “lysate”).

“Disintegration” as used in the present description means, in the context of disintegration of microbial cells, the fragmentation of the cells. This implies that the average size of the resulting cell fragments must be smaller than the average cell size of the initial microbial cells. Disintegration can be seen as a specific type of disrupting in which not only the cells are opened, but in which the cells are also fragmented.

“Cytoplasmic material” or “Cytoplasmic compounds” as used in the present invention means all material that is usually contained within a cell, enclosed by the cell membrane, except for the cell nucleus (if present). When a cell is disintegrated or disrupted, the cytoplasmic material is released from the cell.

“Microbial cells” as used in the present description means: microbes. This can be eukaryotic and prokaryotic unicellular organisms and colonies of them. A prokaryote is a cellular organism that lacks an envelope-enclosed nucleus. In the three-domain system, based upon molecular analysis, prokaryotes are divided into two domains: Bacteria (formerly Eubacteria) and Archaea (formerly Archaebacteria). Organisms with nuclei are placed in a third domain, Eukaryota. Microbial cells according to the present invention also encompass algae and fungi such as yeast.

“Microorganism” and “microbe” as used in the present description mean any microscopic colonial or unicellular organism.

“Microbial cell product” as used in the present description means: a product derived from microbial cells that is obtained by processing microbial cells in a certain manner.

“Light fraction” or “light phase” as used in the present description refers to the phase of a microbial cell extract enriched in small cell fragments (EESF) in the range of around 0.1-3 μm. “Small cell fragments” as used in the present description means cell fragments obtained from disintegration of microbial cells having a size of equal to or less than d50≤500 nanometers (nm). A light and a small fraction are used interchangeably herein.

“Heavy fraction” or “heavy phase” as used in the present description refers to the phase of the microbial cell extract enriched in large cell fragments (EELF) that are at a size of >1 μm. “Large cell fragments” as used in the present description means cell fragments obtained from disintegration of microbial cells having a size more than d50≥500 nanometer (nm). A heavy and a large fraction are used interchangeably herein.

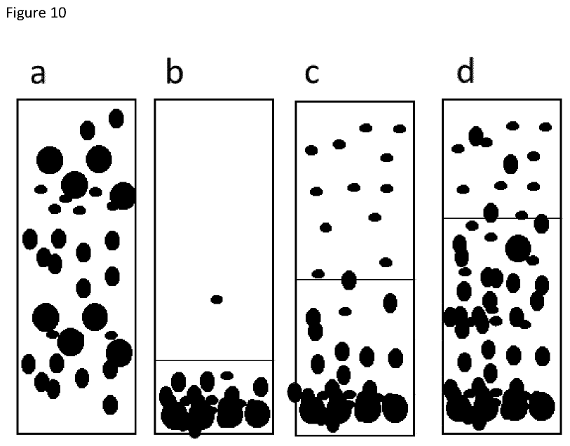

“Enriched” or “enrichment” as used in the present description means selective movement of particles to one of the two phases of separation i.e. the EESF or the EELF; this concept is illustrated in FIG. 10. When using high centrifugal forces, all particles/insoluble material are transferred to the heavy phase (FIG. 10b), if a centrifugal force is used that is too low the separation between the light and the heavy phase is poor and fragments are not clearly separated (FIG. 10d). However, using a mild or medium centrifugal force results in the small fragments being preferentially concentrated in the light phase (extract enriched in small fragments), while large fragments are preferentially concentrated in the heavy phase (extract enriched in large fragments), shown in FIG. 10c. The concept of enrichment is further described in example 2. Note that this is not the same as simply separating soluble and insoluble fractions, since insoluble material remains in both the EESF and EELF.

“Microbial biomass” and “biomass” as used in the present description mean a material produced by growth and/or propagation of microbial cells, or produced as byproduct of fermentation processes. Biomass may contain cells and/or intracellular contents as well as extracellular material. Extracellular material includes, but is not limited to, compounds secreted by a cell.

“Bead milling” as used in the present description means agitation of microbial cells in suspension with small abrasive particles (beads). Cells break because of shear forces, grinding between beads, and collisions with/between beads. Shear forces produced by the beads disrupt the cells and cause disintegration with concomitant release of cellular compounds.

“Centrifugation” as used in the present description means the application of centrifugal force to separate particles from a solution according to their size, shape, density, viscosity of the medium and rotor speed, among other parameters. The rate of centrifugation is specified by the angular velocity usually expressed as revolutions per minute (RPM), or acceleration expressed as g. The conversion factor between RPM and g depends on the radius of the centrifuge rotor. The general formula for calculating the revolutions per minute (RPM) of a centrifuge is

$$R P M = \sqrt{\frac{g}{r}}$$

where g represents the respective force of the centrifuge and r the radius from the center of the rotor to a point in the sample. However, depending on the centrifuge model used, the respective angle of the rotor and the radius may vary, thus the formula gets modified. The most common formula used for calculating Relative Centrifugal Force is:

$$R C F ( ⋆ g ) = 1.118 ⋆ r ⋆ {( \frac{R P M}{1000} )}^{2}$$

wherein r is the radius in mm.

“Feed conversion ratio (FCR)” as used in the present description refers to the conventional measure of livestock production efficiency. Animals that have a low FCR are considered efficient users of feed in the art. The most common formula used to calculate FCR is:

$$F C R = \frac{Total Feed Consumed}{Weight Gain of Animal}$$

“Water holding capacity (WHC)” as used in the present description refers to the amount of water a sample can hold per unit of weight.

“Oil holding capacity (OHC)” as used in the present description refers to the amount of oil a sample can hold per unit of weight.

“Litter quality” as used in the present description means the mix of bedding material, manure and other waste material that result from animal farming

The present invention relates to a method for producing a microbial cell extract and its use in diet formulations for animals. In an embodiment, the invention relates to the use of the microbial extract in diet formulations to decrease feed conversion ratios and/or improve body weight and/or litter quality in animals. In a further embodiment, the invention relates to the use of the microbial extract as a prebiotic in the diet of animals. Moreover, the microbial extract may be further processed according to the present invention to further improve its functional properties, said functional properties include but are not limited to, gelation hardness, water holding capacity and oil holding capacity, as well as β-glucan and RNA content.

The invention relates in a first aspect to a method for preparing a microbial cell extract, said method comprising providing a microbial biomass, subjecting said microbial biomass to disintegration; and separating the disintegrated microbial biomass into two phases enriched in fragments of a small and large size, suspended in an aqueous mixture with soluble compounds. Said method may also optionally comprise a polishing step to improve the purity of the components and/or a concentration and/or drying step.

Microbial biomasses, which comprises microbial cells, have been used traditionally to produce a broad range of products of industrial interest, or have been used directly in a number of applications. Most industrial or commercial applications make use of a selected group of microbial biomass strains from the domains bacteria, yeast, fungi and algae. In overall terms, products obtained from microbial biomass are either intracellular or extracellular. Extracellular products are excreted by the cells into the bulk medium, usually an aqueous phase. Intracellular products, on the contrary, remain inside of the cells. In order to obtain intracellular products, additional processing is needed to release these products from the cells (by breaking the cell membrane or wall) and to further separate the compounds of interest from the remaining biomass and other impurities.

In the particular field of food applications, microbial biomasses have been used as a source of proteins (single cell protein—SCP), as nutritional supplements, or to produce various ingredients and additives.

Microbial biomasses are often used in the form of extracts, for which the microbial cells forming said biomass need to be disrupted/disintegrated. Extracts prepared from several different starting materials are known, such as fungal extract, algae extract and yeast extract. Of these extracts, the most commonly used is that derived from yeast, the so-called yeast extracts (hereinafter referred to as “YE”). YE are (and can be) applied in a broad range of products ranging from growth media for culturing cells for laboratories to nutritional supplements and flavor enhancers for the food industry. Production processes of YE are well known. In general, yeast cells, mostly from the genus Saccharomyces, are disrupted (=disintegrated) by means of heat induced or chemically induced autolysis (or plasmolysis), followed by a step of incubation at high temperatures (>50° C.) in order to activate endogenous enzymes, which break down (=digest) the large intracellular products such as proteins and nucleic acids into smaller components thereof such as peptides, amino acids and nucleotides. The digested slurry that is obtained is then further purified and supplemented—depending on the final application—to provide an extract that can be commercialized as YE.

The microbial biomass used in the present invention can be obtained from several microbial types, including microalgae, yeast, bacteria and fungi. Examples of genus from which the microbial biomass may be derived for the microbial extract may be produced are Saccharomyces and Pichia (yeast), Tetraselmis, Chlorella, Arthrospira (algae), Fusarium (fungi), Methylobacterium (bacteria) and Lactobacillus (bacteria). Preferably the microbial biomass is derived from yeast, more preferably from the genus Saccharomyces and/or Pichia. Yeasts which may be used in the present invention include Saccharomyces, such as S. cerevisiae, S. chevalieri, S. boulardii, S. bayanus, S. italicus, S. delbrueckii, S. rosei, S. micro-ellipsodes, S. carlsbergensis, S. bisporus, S. fermentati, S. pastorianis, S. rouxii, or S. uvarum; a yeast belonging to the genus Schizo-saccharomyces, such as S. japonicus, S. kambucha, S. octo-sporus, or S. pombe; a yeast belonging to the genus Hansenula, such as H. wingei, H. arni, H. henricii, H. americana, H. canadiensis, H. capsulata, or H. polymorpha; a yeast belonging to the genus Candida, such as C. albicans, C. utilis, C. boidinii, C. stellatoidea, C. famata, C. tropicalis, C. glabrata, or C. parapsilosis; a yeast belonging to the genus Pichia, such as P. pastoris, P. kluyveri, P. polymorpha, P. barkeri, P. cactophila, P. rhodanensis, P. cecembensis, P. cephalocereana, P. eremophilia, P. fermentans, or P. kudriavzevii; a yeast belonging to the genus Kluyveromyces, such as K. marxianus; and a yeast belonging to the genus Torulopsis, such as T. bovina, or T. glabrata.

In a preferred embodiment the microbial biomass is free from polluting material—for example, the biomass may be purified by centrifugation followed by washing and resuspension; several rounds of washing and resuspension may be used. In another embodiment said microbial biomass is prepared in an aqueous alkaline suspension (pH ˜9), optionally around 50-100 g/L.

In a preferred embodiment the disintegration step is a method of mechanical disintegration known in the art. More preferably said mechanical disintegration step is performed using bead milling or high-pressure homogenization. Preferably, the disintegration step is carried out at a pH in the range of 6-9, and at a temperature in the range of approximately 10-40° C., preferably 10 to 30° C. and more preferably 20-25° C., and even more preferably around 20° C. Carrying out the disintegration step within the said pH and temperature ranges has the technical effect of preventing the activation of lytic enzymes, proteases or other hydrolytic enzymes.

Generally, cell disintegration methods can be classified as being either non-mechanical or mechanical. Non-mechanical disintegration methods can be further sub classified into three categories: physical disintegration (e.g. by means of decompression, osmotic shock, thermolysis, ultrasonics, or freezing and thawing), chemical disintegration (e.g. by use of solvents, detergents, chaotropes, acids and bases, or chelates) and enzymatic disintegration (e.g. by autolysis, phage lysis, or lytic enzymes). The present invention is preferably related to mechanical disintegration methods. Examples of mechanical disintegration methods are ball mills, including bead mills, and homogenizers.

Ball mills (including bead mills) can be either vertical and horizontal and use a grinding medium which is present in the grinding chamber. A motor drives a rotor to rotate the cell suspension at a high speed. The cell suspension and the grinding material (e.g. beads) generate shearing force to break the cells. This results in the release of intracellular materials into the aqueous suspension and will also result in cell fragmentation (i.e., disintegration). With increasing rotor speed, the shear force increases and the cell breakage increases. With decreasing grinding material size, the cell breakage usually increases. Other parameters affect the performance of the disintegration process. The skilled person is capable of selecting the right parameters and variables in accordance to the present invention.

Homogenizers work under high-pressure and are in fact a positive-displacement pump that forces a cell suspension through a valve, before impacting the stream at high velocity on an impact ring. Often, several passes at high-pressure are required, which may lead to rising temperatures causing local denaturation of labile molecules.

Preferably the mechanical disintegration step is performed using bead milling or high pressure homogenization. Most preferably the mechanical disintegration step is performed using bead milling. Preferably, the disintegration step is carried out at a pH in the range of 7-11 (optionally 8-10, or 8.5-9.5 or most preferably 9), and at a temperature in the range of approximately 10-30° C., more preferably approximately 15-25° C., even more preferably 20-25° C., and most preferably around 20° C. Carrying out the disintegration step within the said pH and temperature ranges has the technical effect of preventing the denaturation of proteins and other labile molecules (that is, it is a non-denaturing process) and consequently preventing the activation of lytic enzymes, proteases or other hydrolytic enzymes present in the microbial biomass. The skilled person is able to adjust the process parameters of the disintegration method (speeds, flows, filling ratios, bead sizes, pressures, etc.) in order to keep the temperature preferably below <25° C. and to reach the desired psd target.

The disintegration step results in the production of a population of soluble compounds and suspended fragments characterized by a bimodal distribution and a particle size distribution (psd) at an average of around D50<4.5 μm. It is recognized that the skilled person would be able to adjust the parameters used during the disintegration step accordingly to achieve the desired targets of their application. During the course of the cell disintegration process, the psd varies, showing a decrease in the peak of intact cells, and a consequent increase in the peak of cell fragments. The disintegration process is run until a specific psd is obtained. Other psd parameters may also be considered; for example, for yeast, one desired target psd may be D10<0.7 um, D50<4.5 um and D90<8.5 um.

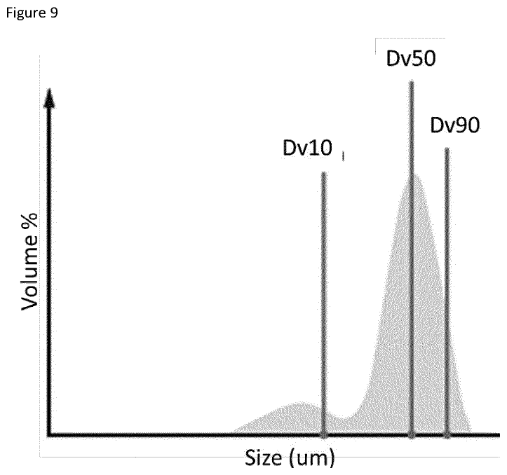

In the art, particle size distribution is reported as a volume distribution. Taking D50 as an example it is known in the art that this can also be referred to as Dv50; the terms D50 and Dv50 are used interchangeably herein. The D50 or Dv50 is defined in the art as the maximum particle size, measured by diameter, below which 50% of the sample volume exists, also known as the median particle size (diameter) by volume. This concept is illustrated in FIG. 9. Numerous analytical techniques and approaches exist for particle size analysis.

A particle size analyser is an analytical instrument that measures, visualises, and reports a particle size distribution for a given particle or droplet population. Laser diffraction particle size analysers calculate particle size from the angle of light scattered by a stream of particles passing through a laser beam. This technique allows for continuous measurement of bulk material across a wide size range. The size limits and sensitivity of a laser diffraction particle analyser depend on the number and placement of detectors in the instrument. Dynamic light scattering particle analysers are mainly used for analysing particles in solution. Dynamic light scattering determines size from the fluctuations in scattered laser light intensity created by the particles' Brownian motion. Induced grating particle size analysers identify the size of small particles in solution by electrically aligning the particles and then measuring their diffusion.

In the context of the present invention the D50 was determined using a laser diffraction particle size analyser, the Malvern Mastersizer 2000, with a dispersant RI of 1.33 and a particle/material RI of 1.34, using a general-purpose analysis Model MS2000, and the Mie scattering model.

Disintegration methods are used to obtain a bimodal distribution and a particle size distribution (psd) at an average of around D50<4.5 μm as described above. Examples of cell disintegration methods according to the prior art are the following. U.S. Pat. No. 3,888,839A discloses a process for obtaining a protein isolate from yeast cells, wherein the yeast cells are ruptured by high-pressure homogenization (mechanical disintegration) and subsequent incubation. EP1199353A1 discloses a process for producing yeast extracts by treating yeast suspensions or yeast pastes and separating off the insoluble constituents, in which the yeast suspensions or yeast pastes are subjected to high-voltage electrical pulses (physical disintegration). EP2774993A1A discloses the use of a cell wall-decomposing enzyme (enzymatic disintegration) that does not contain protease and then heat-treating the product for 10 to 20 minutes at 70-80° C.

Microbial cells present in microbial biomass suspensions contain mostly proteins, carbohydrates, lipids and minerals. Proteins and other labile molecules experience unfolding, denaturation and degradation when exposed to high temperatures, long incubation times, extreme values of pH, solvents, salts and other harsh chemicals. When proteins and other functional molecules are denatured (tertiary and quaternary structure is lost), (part of) their functional activity is lost. Upon denaturation (unfolding), proteins lose their ability to interact with hydrophilic and hydrophobic surfaces, and also their ability to rearrange and form network-like structures upon heat-cooling treatments is affected. The present inventors have observed that the use of mechanical disintegration at the conditions described herein (for example, at the stated pH range) is sufficiently gentle to prevent unfolding, denaturation and/or degradation of proteins and other labile molecules, and therefore, necessary to preserve the functional properties, in particular gelation behavior, water holding capacity and oil holding capacity.

The aqueous suspension comprising microbial biomass may further comprise cytoplasmic material or other extracellular material produced during propagation or fermentation.

In an embodiment of the method according to the present invention, the microbial biomass comprises microbial cells selected from unicellular or colonial prokaryotes and eukaryotes and one or more combinations thereof. In a preferred embodiment, the microbial cells are selected from the group consisting of yeast, algae, bacteria, fungi, and one or more combinations thereof. In a specific embodiment, the microbial cells are yeast.

Bead sizes that may be considered are in the range of 0.1-5 mm, preferably in the range of 0.5-1 mm. Suitable bead materials include, but are not limited to, zirconium and glass. Bead fillings (the percentage of the bead mill chamber that is filled with beads) that may be considered suitable are in the range of 40-90%, preferably in the range of 65-80%, more preferably 75%, based on the total available volume of the bead mill chamber.

Rotational speeds that may be considered suitable are in the range of 1-20 m/s. Depending on the configuration and geometry of each bead mill, the skilled person can estimate the corresponding rotor speeds in rpm. Suitable rotational speeds in rpm are for example 500-5000 rpm, preferentially 1000-3000 rpm.

Concentration of microbial cells that may be considered suitable are in the range of 2-25% dry weight.

In one specific embodiment, microbial cells are disintegrated using a Dyno-mill Research Lab (CB Mills) bead mill. Cells can also be disrupted by shear forces, such as with the use of blending (such as with a high speed or Waring blender as examples), the French press, or even centrifugation in case of weak cell walls, to disintegrate cells.

In an embodiment, cell disintegration takes place without the addition of chemicals and/or solvents.

The disintegration step results in the production of a population of soluble compounds and suspended fragments characterized by a bimodal distribution and a particle size distribution (psd) at an average of around D50<4.5 μm. It is recognized that the skilled person would be able to adjust the parameters used during the disintegration step accordingly to achieve the desired targets of their application.

In a preferred embodiment the separation step (or classification step; such terms may be used interchangeably) is selected from a method known in the art including, but not limited to, centrifugation, decantation and filtration. More preferably, separation is performed by centrifugation, in a most preferred embodiment separation is performed using centrifugation with a mild centrifugal field, an embodiment of the centrifugal field that may be applied is set out in Example 2.

In one embodiment the separation step of the method yields a light fraction comprising small cell fragments in the range of around 0.1-3 μm and a heavy fraction comprising large cell fragments that are at a size of >1 μm. Preferably classification yields a volumetric ratio of heavy fraction to total starting fraction (disintegrated cells) >0.65, preferably >0.9. The skilled person would be able to adjust the separation parameters, for example, centrifugation parameters such as but not limited to any number of, time, g force, and/or sigma factor. It would be recognised by the skilled person that how said parameters are adjusted would vary depending on the centrifugation unit employed to achieve the desired classification target.

In a preferred embodiment the classification step of the present invention, is not employed for the purposes of producing a soluble phase and an insoluble phase. The present invention is instead characterised by the production of two phases each of which is enriched in fragments of different particle sizes, namely a light fraction (enriched in small cell fragments in the range of around 0.1-3 μm) and a heavy fraction (enriched in large cell fragments that are at a size of >1 μm). The light and heavy fractions are suspended in an aqueous mixture comprising soluble compounds.

In an embodiment, the light fraction exhibits enhanced functional properties characterised by enhanced gelation hardness, water holding capacity, oil holding capacity, foaming ability and emulsification stability. This enhanced functionality is demonstrated in Example 7.

The microbial cell extract obtained from the method in the first aspect of the invention whereby a microbial biomass is subjected to disintegration and separation as set out above may subsequently be optionally polished, to further purify the components. In a preferred embodiment the microbial cell extract is polished using technologies and methods known in the art. Such methods include but are not limited to filtration, absorption, isoelectric precipitation, coagulation and solvent extraction. In a preferred embodiment subjecting the microbial cell extract obtained from disintegration and classification to polishing improves the purity of the main components of said microbial cell extract.

In another embodiment, the microbial cell extract may optionally be concentrated and/or dried. In a preferred embodiment the microbial cell extract is concentrated and/or dried using technologies and methods known in the art. Such methods include, but are not limited to filtration, evaporation, freeze concentration, pervaporation, sublimation and drying by spray drying or drum drying.

The concentration and/or drying step may be carried out in the presence or absence of the optional polishing step. When the concentration and/or drying step is carried out in addition to the optional polishing step, the concentration and/or drying step may be performed before or after the polishing step.

In one embodiment the optional concentration and/or drying step reduces the water content of the microbial cell extract. In a preferred embodiment the concentration and/or drying step reduces the water content of the microbial cell extract to approximately 5% of the water content of the microbial cell extract prior to concentration and/or drying.

In another aspect of the invention, there is provided a microbial cell extract, the extract comprising microbial cell fragments between 1 and 15 μm suspended in an aqueous mixture.

In one embodiment the microbial cell extract produced by the method of the first aspect of the invention or the microbial cell extract can absorb approximately twice its own weight in water. In a preferred embodiment said microbial cell extract can absorb more than twice its own weight in water, such as 3 times, 4 times or 5 times. In another embodiment, said microbial cell extract can absorb approximately 1.5 times its own weight in oil. In a preferred embodiment said microbial cell extract can absorb more than 1.5 times its own weight in oil, such as 2 times, 3 times, 4 times or 5 times its own weight in oil. In another embodiment said microbial cell extract can absorb both approximately twice its own weight in water and approximately 1.5 times its own weight in oil. In a preferred embodiment said microbial cell extract can absorb more than twice its own weight in water and more than 1.5 times its own weight in oil.

In one embodiment, the microbial cell extract produced has a β-glucan content around 10 to 50%, more preferably around 15 to 30% by dry weight of dry cell extract. In another embodiment, the microbial cell extract produced has a β-glucan content of >20% by dry weight of dry cell extract. In another embodiment said microbial cell extract has an RNA content <5%, or <4%, or <3% or <2% or <1% by dry weight of dry cell extract. In a preferred embodiment said microbial cell extract has a β-glucan content >20% by dry weight of dry cell extract and an RNA content <5% by dry weight of dry cell extract.

The invention relates in a second aspect to a method of preparing a microbial cell extract with an enhanced β-glucan content, the method comprising firstly preparing a microbial cell extract according to the first aspect of the invention whereby a microbial biomass is subjected to disintegration, classification and optional polishing and/or concentration and/or drying; secondly subjecting the heavy of fraction of said microbial cell extract to further processing by subjecting the heavy fraction of said microbial cell extract to extended disintegration; subsequently diluting the resulting disrupted suspension; subjecting the diluted disrupted suspension to solid-liquid separation; and subsequently removing the light phase and retaining the heavy phase. Also encompassed is a microbial cell extract obtained from or obtainable from the method.

In one embodiment of any of the above aspects, the microbial cell extract comprises a heavy fraction enriched in large cell fragments. In a preferred embodiment said heavy fraction comprises large fragments >1 μm in a further preferred embodiment said heavy fraction comprises large fragments in the range of 1-15 μm. In a further embodiment the volumetric ratio of the heavy fraction to the total is >0.65. in an aqueous mixture. In a further embodiment there is a DW content in the range of 5-15%.

In one embodiment the extended disintegration step comprises a method of mechanical disintegration known in the art. More preferably said extended disintegration step is performed using bead milling for an extended period of time. Preferably, the extended disintegration step is carried out at a pH in the range of 9-11, more preferably at pH 11. Preferably the extended disintegration step is carried out for a period if between 20-500 minutes, more preferably extended disintegration is carried out for 240 minutes.

In one embodiment aqueous dilution of the disrupted suspension resulting from extended disintegration results in a diluted disrupted suspension of around 1 to 10% DW, preferably at least 2.5% DW.

In a further embodiment the diluted disrupted suspension is subjected to solid-liquid separation by a method known in the art, preferably by centrifugation.

In one embodiment the retained heavy phase referred to herein as a microbial cell extract with an enhanced β-glucan content has a high β-glucan content, and preferably said microbial cell extract with enhanced β-glucan content has a low RNA content. In one embodiment the microbial cell extract with an enhanced β-glucan content has a β-glucan content of >40%, in another embodiment the microbial cell extract with an enhanced s-glucan content has an RNA content of <2%. In a preferred embodiment the microbial cell extract with an enhanced β-glucan content has a β-glucan content of >40% and an RNA content of <2%.

In one embodiment the microbial cell extract produced by the method of the first aspect of the invention can absorb approximately 10 times its own weight in water.

In one embodiment the microbial cell extract with an enhanced β-glucan content exhibits enhanced functionality, characterized by increased gel hardness compared to microbial cell extracts with a lower β-glucan content, and/or increased water holding capacity compared to microbial cell extracts with lower β-glucan content, and/or increased oil holding capacity compared to microbial cell extracts with lower β-glucan content. This increased functionality is demonstrated in Example 6.

In one embodiment the microbial cell extract with enhanced β-glucan content produced by the method of the second aspect of the invention is advantageous because it does not require the use of alkaline-acid extractions, intrinsic or external enzyme hydrolysis, thermal treatments or prolonged incubation/processing steps. The method of the second aspect of the invention is also advantageous because it does not require intrinsic biochemical reactions or external addition of enzymes to reduce RNA content.

In one embodiment the microbial cell extract (of the first or second aspect) can be incorporated into diet formulations for animals using methods known in the art including but not limited to, extrusion, palletisation, blending, and coating amongst others. Said microbial cell extract may be incorporated into diet formulations for livestock, including animals including but not limited to, poultry, fish, cattle, pork, sheep, horse, dogs, cats, birds or any other any other animal. Accordingly, in another aspect of the invention there is provided an animal diet formulation comprising or consisting of the microbial cell extract of the invention. In one embodiment, the animal may be a human. In another embodiment, the animal may be any animal other than a human. Preferably the animal is livestock e.g. farm animals including poultry. The composition of a typical diet formulation for poultry is shown in Table 6. Other diet formulations four poultry and other livestock would be well known to the skilled person. In one embodiment, a feed formulation may comprise a source of water, lipids and fatty acids, proteins and amino acids, carbohydrate, energy, minerals and vitamins. For example, the feed formulation may comprise one or more of the following ingredients: Corn, Soybean meal, Dried and wet distillers' grains, Bakery meal, Corn gluten feed, Cottonseed meal, Wheat midds, Grain sorghum, Soybean hulls, Oats, Amino acids, Vitamins, Minerals, Probiotics, Enzymes, Animal protein products, Fats and oils, Marine products, Milk products, wheat products and Flavors.

In one embodiment the microbial cell extract produced by the method of the invention can decrease feed conversion ratios (FCR) and increase weight gain in animals compared to the levels than are generally obtained with conventional feeds. This effect may be observed when said microbial cell extract is used in diet formulations at inclusion levels >0.01% by weight. In one embodiment, a reduced FCR is achieved when said microbial cell extract is used in diet formulation at inclusion levels >0.01% by weight and are comparable to commercial products that are currently available with a β-glucan purity (>50%). In a preferred embodiment the FCRs achieved when said microbial cell extract is used in diet formulation at inclusion levels >0.01% by weight are superior to commercial products that are currently available with a β-glucan purity (>50%). In one embodiment, weight gain may be increased by at least 10%, or at least 20% or at least 30% or at least 40% or at least 50% compared to the weight of animals that are not fed the extract or formulation of the invention. In another embodiment, FCR may be decreased by at least 5%, 10%, 15%, 20%, 25%, 30%, 35% or 40%, preferably around 15% compared to the FCR of animals that are not fed the extract or formulation of the invention. Accordingly, in a further aspect of the invention, there is provided a method of decreasing feed conversion rates and/or increasing the body weight of an animal, the method comprising feeding the microbial cell extract or animal feed formulation to the animal. In one embodiment, the animal is not a human. In another embodiment, the animal may be selected from poultry, fish, cattle, pig, sheep, horse, dog, cat and bird.

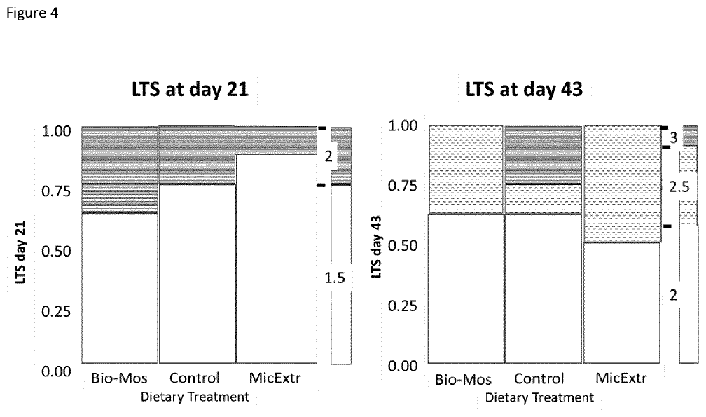

In another embodiment, the microbial cell extract or the microbial cell extract produced by the method of the first aspect of the invention improves the litter quality of animals fed with a diet that includes at least 0.01% by dry weight of said microbial cell extract. This is demonstrated in Example 5. The quality of litter may be assessed by an expert who gives the litter quality a score, for example as shown in FIG. 4, where 1=dry compact, 2=wet compact, 3=soaked compact, 3.5=sticky soaked. FIG. 4 shows that the litter quality improves, resulting in dry compact or at least wet compact (scale 1-2.5) in comparison to the control, where the litter quality starts becoming soaked and sticky soaked (score 3-3.5). Improved litter quality (drier and more compact litter) is advantageous as this means that animals are less effected by at least one of the following factors, humidity, microbial decomposition, smells, ammonia generation, injuries in their feet and/or minimizing the risks of infections and illness. In particular, it is well known in the field that dry, compact litter is most desirable as the drier and more compact the litter, the less sole ulcers or footpad lesions develop on the animal. In a particular embodiment, the microbial cell extract is particularly useful during the first growth phase of the animal.

Accordingly, in a further aspect of the invention, there is provided the use of the microbial cell extract or the microbial cell extract produced by the method of the invention as a prebiotic. There is also provided a method of improving the litter quality of an animal, the method comprising feeding the microbial cell extract of the invention or the animal feed formulation of the invention to the animal.

In one embodiment the microbial cell extract produced by the method of the invention is advantageous because it does not require the use of alkaline-acid extractions, intrinsic or external enzyme hydrolysis, thermal treatments or prolonged incubation/processing steps. The method of the invention is also advantageous because it does not require intrinsic biochemical reactions or external addition of enzymes to reduce RNA content.

Although primarily described herein with reference to preparations obtained from yeast cells, the invention is not limited to the same. Various other microorganisms can be used. In embodiments, the microbe may be selected from fungi, including yeast (preferably Saccharomyces sp, more preferably brewer's or baker's yeast, or Pichia sp); plants, in particular microalgae (including Tetraselmis sp or Chlorella sp, for example C. vulgaris); and cyanobacteria (including Arthrospira sp, preferably A. platensis). The microbe may also be selected from bacteria, for example Methylobacterium or lactic acid bacteria.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measured cannot be used to advantage.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

### EXAMPLES

### Example #1: Cell Disintegration

A yeast suspension of the genus Saccharomyces, free of foreign contaminants, is subjected to bead milling under the following conditions:
Biomass suspension at ˜100 g/L, pH adjusted from ˜4.5-5.5 to ˜9 using NaOH.
The suspension is bead milled at ˜20° C., 14 m/s tip speed, 75% bead filling, 0.5-1 mm zirconium beads, under batch recirculation mode.
The Temperature of the final disrupted suspension is ˜23° C., and the final pH is ˜6.5.

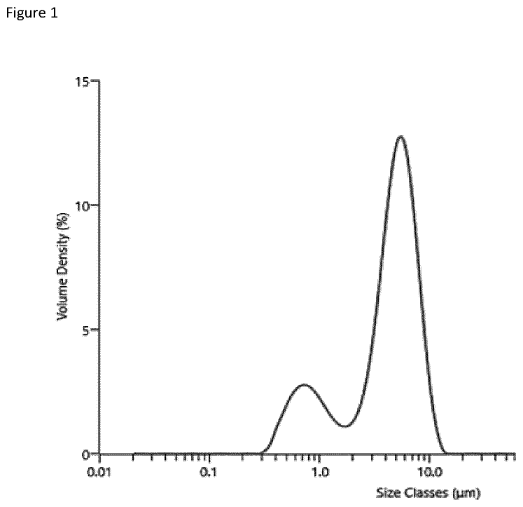

The resulting particle size distribution (psd) is as follows (Table 1 below and FIG. 1):

| D10 | D50 | D90 |
|---|---|---|
| (μm) | (μm) | (μm) |
| 0.697 | 4.56 | 8.21 |

### Example #2: Classification Step

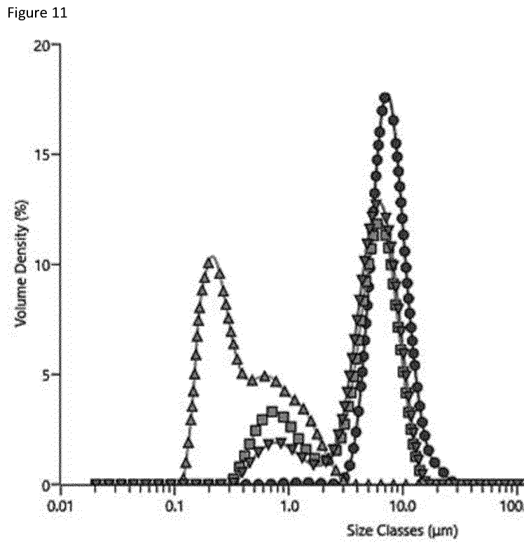

In an example demonstrating enrichment, a microbial biomass with a D50˜7.49 μm (depicted by the circles in FIG. 11) is disintegrated into a suspension with a bimodal distribution, represented by the squares in FIG. 11 with a D50 of ˜4.43 μm. The disintegrated biomass is subsequently separated into an extract enriched in small fragments, represented by the triangles with a D50˜0.35 μm, and a fraction enriched in large fragments, represented by the inverted triangles (FIG. 11) with a D50˜5.41 μm. Therefore, the light fraction will be enriched in small fragments of sizes in the range 0.1-3 μm (D50<0.5 μm. Accordingly, the heavy fraction will be enriched in large-fragments of sizes >0.3 μm (D50>0.5 μm).

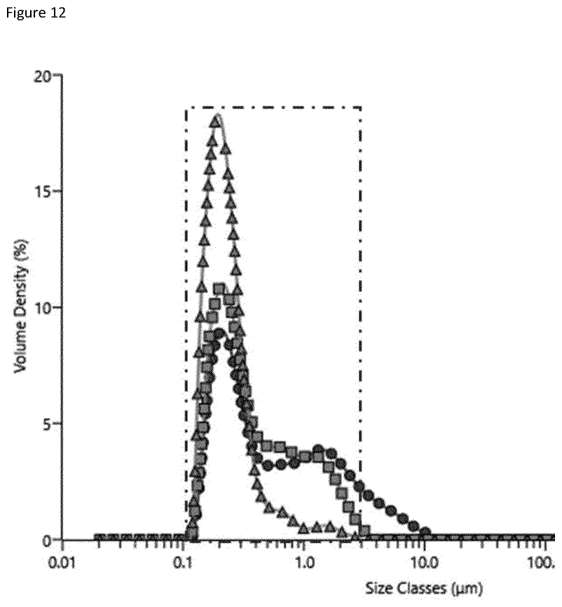

It has been shown that there is a range of centrifugal forces that result in optimal functionality of the fraction enriched in small fragments. FIG. 12 shows a medium centrifugal force leads to superior enrichment of the fragments in the range of 0.1-3 μm, represented by the squares. Strong centrifugal forces (triangles) lead to a psd in the range of 0.1-0.4 μm, while low centrifugal forces result in a psd with fragments spanning to 10 μm (circles). The desired enrichment of small fragments is represented by the dashed square. If centrifugal forces are used which are too high or too low, this leads to a different psd and surprisingly worse functional performance, highlighting that obtaining a psd as defined by the present invention is critical.

Using different centrifugal forces effects the dry matter content of the light and heavy phase. Samples were produced according to the method of the invention as described above, but separated using mild and high centrifugal forces. Following separation these samples were subjected to the oven method known in the art, wherein the samples are kept at 100° C. in an oven until they are at a constant weight. The results of this are shown in table 1. A significant difference was observed in the dry matter content when different centrifugal forces were used. When using a mild centrifugal force of 4000×g for 15 minutes there was additional dry matter in the light phase, due to there being more small particles that remain in suspension. This is an advantage of using a mild centrifugal force to achieve separation.

TABLE 1
Dry matter contents of the light and heavy phase
after centrifugal separation of disrupted biomass
using high and mild centrifugal forces.

| | Dry |
|---|---|
| | Weight % |
| High Centrifugal force: | |
| 20000 xg 15 min | |
| Light phase | 3.9% |
| Heavy phase | 17.2% |
| Mild Centrifugal force: | |
| 4000 xg, 15 min | |
| Light phase = FESF | 6.1% |
| Heavy phase FELF | 13.5% |

In optimised classification a disrupted yeast suspension, having a psd as described in Example #1, is subjected to separation via centrifugation using a bench centrifuge at 4000×g for 10 min. After centrifugation, a light phase and a heavy phase is obtained, with the following characteristics
A volumetric split factor of 75%, measured as the volume light phase to total volume
A light phase having a multimodal psd in the range 0.1-3 um, with mean peaks at 0.2 um and a solids content of ˜50 g/L.
A heavy phase having a multimodal psd in the range 1-15 um, with a main peak at ˜5 um and a solids content of ˜150 g/L.

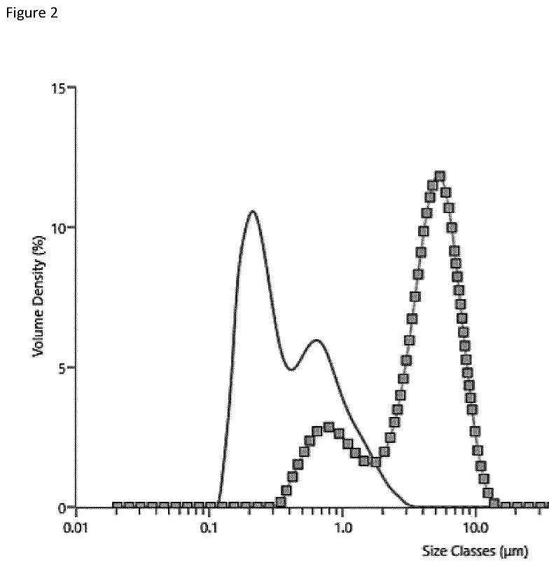

The psd are as follow (Table 2 below and FIG. 2):

| | Dx (10) | Dx (50) | Dx (90) |
|---|---|---|---|
| Phase | (μm) | (μm) | (μm) |
| Light | 0.171 | 0.337 | 1.11 |
| Heavy | 0.786 | 4.33 | 7.71 |

The heavy phase is subjected to centrifugation at 4000×g for 15 min, resulting in a sub light phase and sub heavy phase, in which the volumetric ratio of sub phases is 0.95, calculated as follows:
   The centrifugation test in done in 15 ml tubes, using 10 ml of feed (V_{f}).
   The volume of the sub heavy phase (Vₛₕₚ) is ˜9.5 ml while the volume of the light phase is ˜0.5 ml, thus the volumetric ratio is Vₛₕₚ/V_{f}=9.5/10=0.95

### Example #3: Composition and Functional Properties of the Microbial Extract

The microbial extract obtained as described in example #1 is characterized using standard methods and the following results are obtained (Tables 3 and 4):

Composition: % DW

| Protein | Carbs | Fat | Ash | RNA | g Glucans |
|---|---|---|---|---|---|
| 36.7 | 43.4 | 5.1 | 5.8 | 3.9 | 14.7 |

Functionalities

| WHC [g/g] | OHC [g/g] | Gel hardness [N] |
|---|---|---|
| 2.5 | 1.6 | 0.08 |

TABLE 4
Example dietary fiber content of the microbial extract 1

| Composition | % DW |
|---|---|
| Ins HMW | 8.6 |
| Sol HMW | 24.4 |
| LMW | 1.0 |
| Total DF | 34.0 |

(Ins HMW: Insoluble molecular weight, Sol HMW: Soluble high molecular weight, LMW: low molecular weight, DF: Dietary fiber)

### Example #4: Feed Trials Fish

The purpose of the trials is to evaluate if feeds containing the microbial extract of the present invention will enhance the production, health, and microbiome of aqua-cultured fish. For this study, Rainbow trout was used as a model species for salmonids and finfish in general.
The trials had a duration of 8 weeks.
Trials were conducted in 12-170 tanks, with cold water recirculation and filtration+UV disinfection systems. Fish were stocked at 14 per tank.
The control diet was a commercial feed: Ziegler Bros., Finfish Silver Semi-floating.
The treatment diets were based on the same control diet, coated with the microbial extract at two inclusion levels: low (2%) and high (5%).
Fish survival rates were recorded daily, fish were weighted every two weeks, feed quantities were adjusted daily, and the water quality parameters were rigorously monitored on a daily basis.
Experiments were conducted in 4 replicates (control and treatments) and analysed with a confidence interval of 1% (P<0.01).
At the end of the experiment, the results clearly indicate that the microbial extract had a significant effect on the weight gain (over 20% at low dosages and over 35% at high dosages), and on the feed conversion ratios (˜15% lower). No significant differences were observed between the low and high inclusion levels for the FCR.

TABLE 5

| | Overall | Weight Gain | Percent | |
|---|---|---|---|---|
| | Survival | Mean ± Standard | Difference | |
| Treatment | [%] | Error | Over Control | FCR |
| Control | 98.2 | 56.2 ± 2.5 | | 1.19 ± 0.05 |
| LY | 100.0 | 67.9 ± 0.5** | 20.8 | 1.01 ± 0.02 |
| HY | 100.0 | 76.1 ± 0.4** | 35.4 | 1.04 ± 0.05 |

Furthermore, excellent fillet yields were recorded for the low and high inclusion levels, and comparable to those from the control group. Moreover, Viscerosomatic index (VSI) and hepatosomatic (HSI) indexes were considered normal for all treatment groups.

### Example #5: Feed Trials Poultry

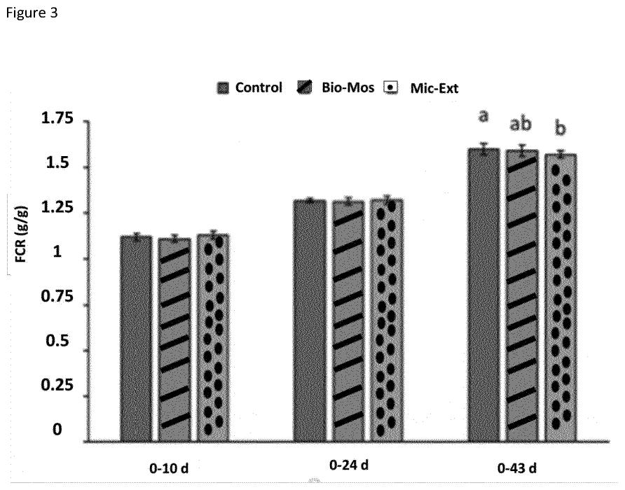

Three experimental diets were investigated in broilers. The basal diet contained corn and wheat as major sources of energy and soy as a major protein source. The protein and energy value of the diets were reduced by 10% compared to recommended values, and no carbohydrases were supplemented.
The basal diet was non-supplemented (negative control), supplemented with commercial b-glucans Bio-Moss from Alltech, USA (positive control), and microbial extract as described in example 1 (Diet) both at a dosage rate of 1 g ingredient per kg of completed feed (0.1%).
Broilers were fed in three phases: a starter (days 0-10), a grower (days 10-24) and a finisher phase (days 24-43). Diets were provided in the form of pellets for starter and grower and finisher, and as crumble crushed pellets for the young chicks.
The trials included 480 male one-day old chicks and used in a completely randomized design with 3 dietary treatments and 8 replicates of 20 broilers each.
All broilers received water and feed ad libitum and were kept under conventional conditions according to the broiler management standards. Housing conditions, water, and feed supply, as well as chicken behaviour, condition and mortality, were checked daily
Feed conversion ratio (FCR) was calculated and corrected for mortality. The cumulative performance (BWG, FI and FCR) was calculated for the period days 0-24 and days 24-43.
Litter score was assessed by an experienced technician four times during the course of the trial. The scores are 1: dry compact litter, 2: wet compact litter, 2.5: wet compact litter, 3: soaked compact litter, and 3.5: sticky soaked litter.
Data was analyzed using one-way ANOVA, while significance was assessed with a poshoc Tukey's HSD test at a significance level of 5% (P<0.05).
Diets containing the microbial extract (MicExt) from the present invention resulted in the lowest FCR (FIG. 3). Diet containing Bio-MOS was significantly comparable to the control diet and the MicExt diet. The results suggest that the effect of the MicExt diet is only evident after a sufficient development time has been achieved, time that is linked to the development of the gastrointestinal microbiota and mucosal architecture.
A significantly lower litter score was observed (FIG. 4) for diets containing MicExt during the first growth phase (21 days) compared to the control and the experimental diet containing Bio-Mos. At the end of the trial (day 43) both Bio-Mos and MicExtr diets resulted in significantly lower litter scores compared to the control. Lower litter score means in overall better conditions during the growth phases and thus, other performance indicators are expected to be improved.

An example of a diet formulation is shown in Table 6:

TABLE 6
Example diet formulations for poultry

| Ingredients, g/kg | Starter | Grower | Finisher |
|---|---|---|---|
| Wheat | 250 | 400 | 500 |
| Corn | 357 | 272 | 202 |
| Soybean meal (46.5% CP) | 280 | 230 | 185 |
| Rapeseedmeal (34.0% CP) | 40.0 | 50.0 | 60.0 |
| Soya hulls | 20.0 | 0.00 | 0.00 |
| Soybean oil (37.5 MJ) | 13.5 | 15.5 | 10.0 |
| Premix ¹ | 5.00 | 5.00 | 5.0 |
| Lime fine | 17.2 | 13.9 | 13.0 |
| Monocalciumphosphate | 7.00 | 4.00 | 1.50 |
| Salt | 2.10 | 2.00 | 1.90 |
| Sodium carbonate | 2.60 | 2.30 | 2.00 |
| L-lysine HCl | 2.05 | 2.30 | 2.45 |
| DL-Methionine | 2.30 | 2.00 | 1.70 |
| L-Threonine | 0.70 | 0.75 | 0.75 |
| L-valine | 0.20 | 0.25 | 0.25 |
| Phytase (Axtra PHY, 500 FTU) | 0.05 | 0.05 | 0.05 |
| Calculated nutrient content, g/kg | | | |
| Metabolisable energy, kcal/kg | 2704 | 2801 | 2850 |
| DM | 878 | 875 | 874 |
| Crude protein | 207 | 195 | 184 |
| Crude fat | 43 | 42 | 49 |
| Crude fibre | 34 | 28 | 29 |
| Carbohydrates | 539 | 563 | 572 |
| Calcium | 9.0 | 7.2 | 6.4 |
| Total phosphorus | 5.6 | 4.9 | 4.2 |
| Available phosphorus | 4.6 | 3.9 | 3.3 |
| Digestible lysine, % | 1.04 | 0.98 | 0.90 |
| Digestible leucine, % | 1.41 | 1.29 | 1.16 |
| Digestible methionine + cysteine, % | 0.77 | 0.72 | 0.67 |
| Digestible arginine, % | 1.18 | 1.07 | 0.97 |
| Digestible threonine, % | 0.68 | 0.63 | 0.58 |
| Digestible tryptophan, % | 0.21 | 0.20 | 0.18 |

### Example #6: Extended Cell Disintegration

The cell disintegration step can be further extended in order to enhance the functional properties of the heavy fraction as described in example #2. This fraction has been described in PCT/EP2021/075137 (WO2022/058287) as an extract enriched in large fragments (EELF). As an example, the psd can be reduced over a period of 240 min in order to yield a EELF with superior gel hardness, water holding capacity WHC and oil holding capacity OHC.

TABLE 6

| Time | D | Dx | | Microbial extract # 2 | | |
|---|---|---|---|---|---|---|
| [min] | [3, 2] | (50) | pH | gel hardness [N] | WHC [g/g] | OHC [g/g] |
| 0 | 5.1 | 5.37 | 9.12 | 0.063 | 3.47 | 1.12 |
| 60 | 1.68 | 3.21 | 6.75 | 0.185 | 4.76 | 1.86 |
| 180 | 0.559 | 1.26 | 6.53 | 0.4724 | 5.08 | 2.17 |
| 240 | 0.531 | 1.16 | 6.45 | 0.6082 | 5.99 | 3.44 |

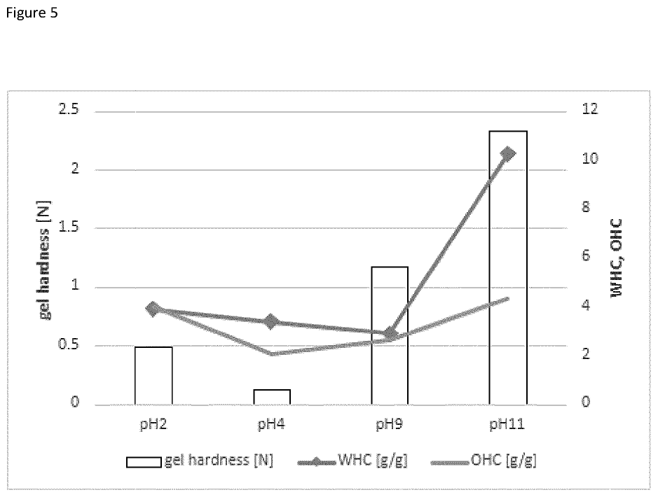

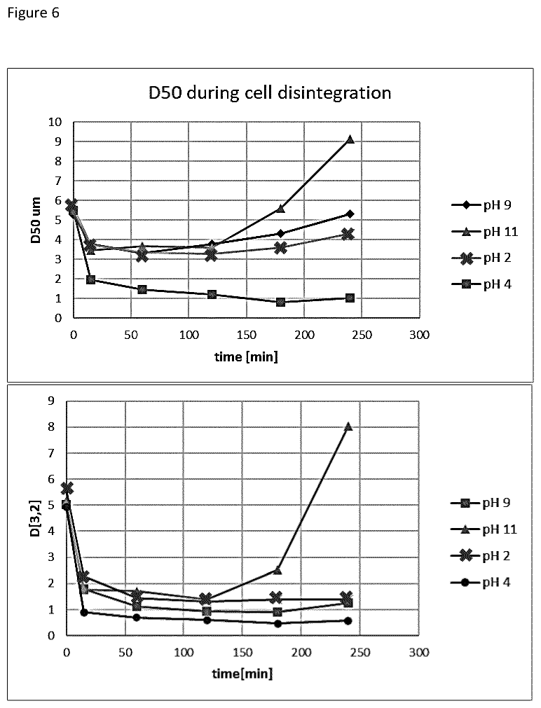

By performing the cell disintegration at alkaline conditions, preferentially at a pH>9, and by conducting the by solid-liquid separation (S/L) at low DW contents (<2.5%), the functional properties of the EELF are significantly enhanced (FIG. 5)
At alkaline pH, however, the psd during the course of extended cell disintegration shows a unique trend. Instead of a steady decrease of the particle sizes, expected for a micronization process, the particles tend to aggregate. This effect is particularly noticeable at pH 11 (FIG. 6)
The extended cell disintegration process conducted at pH 11, followed S/L at high dilution levels (DW<2.5%) produces a EELF with superior functionality in comparison to traditional β-glucan extraction processes applied for yeast biomass. As a reference, a process involving cell autolysis, cell homogenisation, alkaline extraction, acid extraction and aqueous extraction (Aut+Hom+Extr) was compared to the current proposed method (extended disintegration at pH11 and high dilution):

TABLE 7

| | gel hardness [N] | WHC [g/g] | OHC [g/g] |
|---|---|---|---|
| Aut + Hom + Ext | 1.69 | 8.3 | 2.55 |
| pH11 | 2.33 | 10.24 | 4.33 |

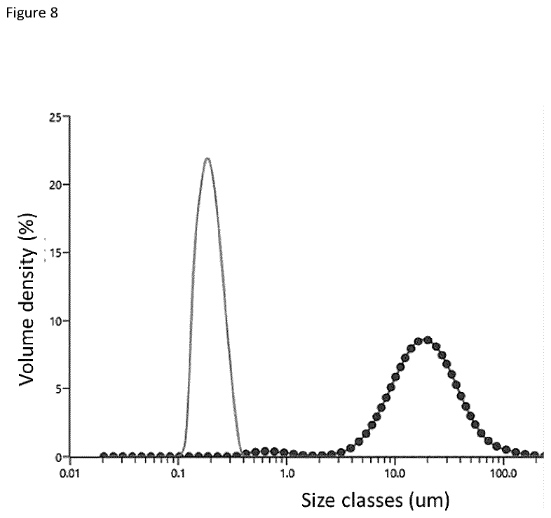

The psd of the heavy and light phase after the extended bead milling at pH11, a high dilution and using solid liquid separation is shown in Table 8 below and FIG. 8.

TABLE 8

| | Dx (10) | Dx (50) | Dx (90) |
|---|---|---|---|
| psd | (μm) | (μm) | (μm) |
| Light phase | 0.142 | 0.193 | 0.274 |
| Heavy phase | 6.95 | 18.2 | 44.9 |

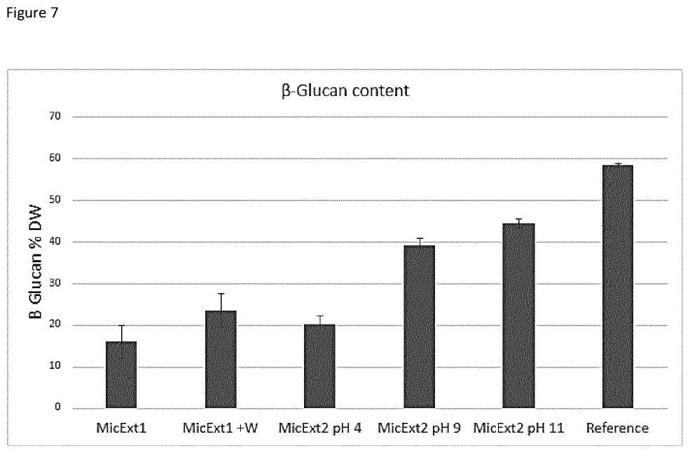

The β-glucan content of the microbial extract described in example #1, and that of the microbial extract obtained after extended disintegration at different pH values was measured and compared to a sample produced following the reference process Aut+Hom+Extr (FIG. 7). These results show that, despite having lower β-glucan content than the reference, the microbial extract, produced using a much simpler process, is still highly functional (e.g. has an improved litter quality).

### Example #7: Functional Properties of the Light Phase

The light phase as described in examples #1 and #2 was collected and dried. The resulting powder was analysed for its functionality, specifically gelation hardness, water holding capacity, oil holding capacity, foaming ability and emulsion stability were measured. The results of this analysis are shown below in table 9. Similar to the heavy phase, the unique processing steps of milling time, pH and dilution prior to separation also resulted in enhanced functionality of the light phase. Notably, these properties were improved compared to those described in PCT/EP2021/075137 (WO2022/058287).

TABLE 9
Functional properties of the light phase
described in examples 1 and 2.

| pH during | | Gel | WHC | OHC | Foam (½ | Emulsion |
|---|---|---|---|---|---|---|
| milling | | hardness [N] | [g/g] | [g/g] | time) [min] | stability |
| pH | 11 | 0.3 | 3.2 | 1.3 | 15 | Very Stable |
| pH | 9 | 0.1 | 2.6 | 1.0 | 3 | Stable |
| pH | 4 | NA = No gel | 0.9 | 1.4 | 10 | Non-Stable |
| pH | 2 | NA = No gel | 0.8 | 1.3 | 1 | Non-Stable |
| Ref, pH | 9 | 0.21 | 1.1 | 1.1 | 2 | Stable |

### Example #9: Functional Properties of the Light and Heavy Phase Obtained from Methylobacterium spp

A microbial cell extract was prepared according to the method described in PCT/EP2021/075137 (WO2022/058287). In summary, said microbial cell extract is produced by i) providing an aqueous suspension comprising microbial cells; ii) subjecting said suspension to mechanical cell disintegration, to obtain an aqueous suspension comprising disintegrated microbial cells; and iii) separating the suspension to provide an extract enriched in small cell fragments (“light fraction”), and an extract enriched in large cell fragments (“heavy fraction”). It is noted that optionally at least a portion of each extract may be recombined, to provide a recombined microbial cell product.

In this example the aqueous suspension comprising microbial cells comprises biomass of Methylobacterium spp. at ˜100 g/L adjusted to ˜pH 9 with NaOH and subjected to cell disintegration via bead milling using 0.3 mm Zirconium beads, with a 65% filling rate, agitation speeds of 2039 rpm, and a temperature of ˜20° C.

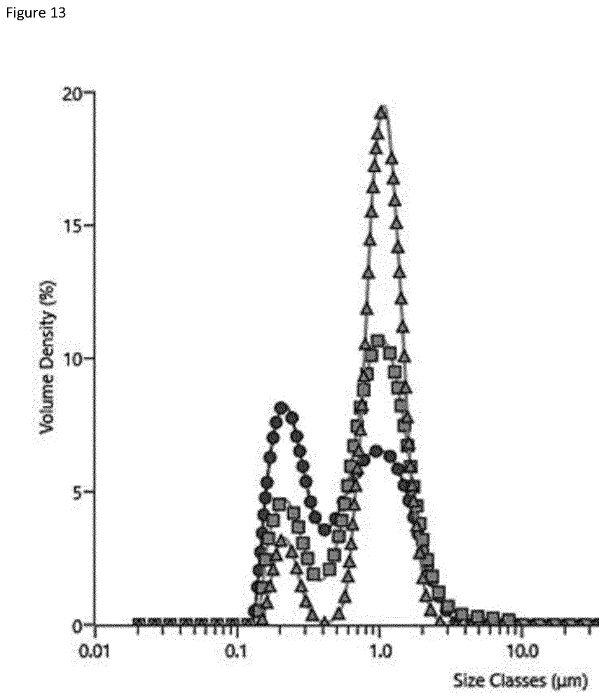

The resulting disintegrated biomass particle size distribution is shown in FIG. 13, and represented by triangles, here it can be seen that a D50˜1.03 μm was achieved.

After the disintegration step, the resulting microbial suspension was subjected to centrifugation using a batch centrifuge at 4000×g for 15 minutes at 15° C. This results in the formation of a light phase and a heavy phase of the microbial suspension (also referred to as an extract enriched in small cell fragments, and an extract enriched in large cell fragments, respectively).

The particle size distribution and particle sizes of the light and heavy fractions resulting from separation by centrifugation are shown in FIG. 13 where the squares represent the heavy fraction and the circles represent the light fraction. The light fraction had a D50 of ˜0.57 μm. Meanwhile, the heavy fraction had a D50˜0.9 μm.

The resulting fractions were then analysed according to their functional properties as shown in table 10 below. Here it is shown that the oil holding capacity and gelation performance were substantially improved in both the EESF and EELF compared to the disintegrated biomass.

TABLE 10
Functional properties of disintegrated Methylobacterium
spp and the resulting EESF and EELF

| Functional | Disintegrated | | |
|---|---|---|---|
| Property | Biomass | EESF | EELF |
| WHC [g/g] | 4.71 | 4.75 | 3.85 |
| OHC [g/g] | 2.23 | 3.15 | 4.61 |
| Gel | No, viscous paste | Yes | Yes |
| G′max [Pa] | 1.19 × 10⁵ | 1.17 × 10⁶ | 8.58 × 10⁵ |

1. A method of preparing a microbial cell extract, the method comprising:
a) providing a microbial biomass in an aqueous alkaline suspension;
b) mechanically disintegrating the microbial biomass at a temperature below 35° C. to produce a disintegrated biomass, wherein the disintegrated biomass consists of a population of soluble compounds and suspended fragments characterized by a bimodal distribution and a particle size distribution (psd) at an average of around D50<4.5 μm;
c) separating the disintegrated biomass into a light fraction and a heavy fraction comprising fragments of different sizes suspended in an aqueous mixture, wherein the light fraction comprises fragments in the range 0.1-3 μm and the heavy fraction comprises fragments above 1 μm, wherein the volumetric ratio of the heavy fraction to the starting disintegrated biomass is 0.65 or more; and
d) selecting only said heavy fraction.

2. The method of claim 1, wherein the microbial biomass is prepared in an aqueous alkaline suspension at a pH of around 9.

3. The method of claim 1, wherein the mechanical disintegration step is carried out by bead milling or high-pressure homogenization, wherein preferably the disintegration step is carried out at a pH in the range of 6 to 9, and at a temperature in the range of 10-30° C.

4. (canceled)

5. The method of claim 1, wherein the disintegrated biomass is separated using centrifugation, decantation or filtration, wherein preferably the disintegrated biomass is separated using centrifugation at around 4000×g for around 10 minutes.

6. (canceled)

7. The method of claim 1, wherein the disintegrated biomass is separated into a light fraction having a multimodal psd in the range of 0.1 to 3 μm and preferably a heavy fraction preferably having a multimodal psd in the range of 1 to 15 μm, wherein preferably the heavy fraction comprises fragments above 1 μm, wherein the volumetric ratio of the heavy fraction to the starting disintegrated biomass is >0.9.

8-9. (canceled)

10. The method of claim 1, wherein the microbial cell extract has a water holding capacity of at least twice its own dry weight and/or an oil holding capacity of at least 1.5 times its own dry weight.

11. (canceled)

12. The method of claim 1, wherein the microbial cell extract has a dry weight β glucan content of 20% and above and/or a dry weight RNA content of 5% or less, wherein preferably the microbial cell extract has a dry weight protein content between 30-60%, more preferably around 36% and/or a dry weight carbohydrate content of about 30 to 60%, more preferably around 43% and/or a dry weight dietary fiber concentration between 30 and 40%, more preferably around 34%.

13-14. (canceled)

15. The method of claim 1, wherein the method further comprises
mechanically disintegrating the heavy fraction, wherein disintegration is carried out at a pH in the range of 9 to 11, preferably a pH of around 11;
diluting the disintegrated heavy fraction to obtain a dry weight (DW) content of 2.5% or more;
subjecting the disintegrated and diluted heavy fraction to a solid-liquid separation process to separate the disintegrated heavy fraction into a first fraction with a particle size distribution (psd) at around D90<0.27 μm and a second fraction with a particle size distribution (psd) at an average of around D50>18 μm; and
selecting said second fraction.

16. The method of claim 15, wherein the microbial cell extract has a water holding capacity of at least 10 times its own dry weight and preferably a gel hardness of at least 2N.

17. The method of claim 15, wherein the microbial cell extract has a dry weight β-glucan content of 40% and above and/or has a dry weight RNA content of 2% or less.

18-19. (canceled)

20. The method of claim 1, further comprising subjecting the microbial cell extract to one or more additional processes, selected from filtration, adsorption, isoelectric precipitation, coagulation and solvent extraction.

21. The method of claim 1, further comprising concentrating and/or drying the microbial cell extract to reduce the water content to around 5% wherein said concentrating and/or drying is preferably carried out by filtration, evaporation, freeze concentration, pervaporation, sublimation, drying by spray drying or drum drying.

22. (canceled)

23. The method ofany-preceding claim 1, wherein the microbial biomass comprises microalgae, yeast, bacteria or fungi; preferably wherein the biomass comprises an organism selected from Saccharomyces, Pichia, Tetraselmis, Chlorella, Arthrospira, Fusarium, Methylobacterium and Lactobacillus.

24. A microbial cell extract obtained or obtainable by the method of claim 1.

25. A method for preparing an animal diet formulation, the method comprising combining the microbial cell extract of claim 24 in a diet formulation using extrusion, palletization, blending or coating, wherein preferably the animal diet formulation comprises 0.01% by weight or more of the microbial cell extract.

26. (canceled)

27. An animal diet formulation obtained or obtainable by the method of claim 25.

28. A microbial cell extract, the extract comprising microbial cell fragments between 1 and 15 μm suspended in an aqueous mixture, wherein the microbial cell extract is characterized by a dry weight β-glucan content of 20% or more and a dry weight RNA content of 5% or less.

29. (canceled)

30. The microbial cell extract of claim 28, wherein the extract is characterized by a dry weight β-glucan content of 40% or more and a dry weight RNA content of 2% or less; wherein preferably the extract has a water holding capacity of at least twice its own dry weight, more preferably at least ten times its own dry weight, and/or an oil holding capacity of at least 1.5 times its own dry weight.

31-33. (canceled)

34. An animal diet formulation comprising 0.01% by weight or more of the microbial cell extract of claim 28.

35. (canceled)

36. A method of decreasing feed conversion rates and/or increasing the body weight of an animal, the method comprising feeding the microbial cell extract of claim 28 or an animal feed formulation comprising said microbial extract to the animal wherein preferably the animal is livestock, including poultry and wherein preferably body weight is increased by 20% or more.

37. A method of increasing the litter quality of an animal, the method comprising feeding the microbial cell extract of claim 28 or an animal feed formulation comprising said microbial cell extract to the animal, wherein preferably the animal is livestock, including poultry.

38-40. (canceled)
